SEQUENCE LISTING (58 sequences)

SEQ ID NO: 1 - protein, 12 aa, Artificial Sequence
  Skin-Binding Peptide
TPFHSPENAPGS

SEQ ID NO: 2 - protein, 12 aa, Artificial Sequence
  Skin-Binding Peptide
KRGRHKRPKRHK

SEQ ID NO: 3 - protein, 7 aa, Artificial Sequence
  Skin-Binding Peptide
RLLRLLR

SEQ ID NO: 4 - protein, 12 aa, Artificial Sequence
  Skin-Binding Peptide
HKPRGGRKKALH

SEQ ID NO: 5 - protein, 18 aa, Artificial Sequence
  Skin-Binding Peptide
KPRPPHGKKHRPKHRPKK

SEQ ID NO: 6 - protein, 18 aa, Artificial Sequence
  Skin-Binding Peptide
RGRPKKGHGKRPGHRARK

SEQ ID NO: 7 - protein, 12 aa, Artificial Sequence
  Skin-Binding Peptide
KQATFPPNPTAY

SEQ ID NO: 8 - protein, 12 aa, Artificial Sequence
  Skin-Binding Peptide
HGHMVSTSQLSI

SEQ ID NO: 9 - protein, 7 aa, Artificial Sequence
  Skin-Binding Peptide
LSPSRMK

SEQ ID NO: 10 - protein, 7 aa, Artificial Sequence
  Skin-Binding Peptide
LPIPRMK

SEQ ID NO: 11 - protein, 7 aa, Artificial Sequence
  Skin-Binding Peptide
HQRPYLT

SEQ ID NO: 12 - protein, 7 aa, Artificial Sequence
  Skin-Binding Peptide
FPPLLRL

SEQ ID NO: 13 - protein, 8 aa, Artificial Sequence
  Caspase 3 Cleavage Site
LESGDEVD

SEQ ID NO: 14 - protein, 37 aa, Artificial Sequence
  Peptide Spacer
TSTSKASTTTTSSKTTTTSSKTTTTTSKTSTTSSSST

SEQ ID NO: 15 - protein, 22 aa, Artificial Sequence
  Peptide Spacer 2
GQGGYGGLGSQGAGRGGLGGQG

SEQ ID NO: 16 - protein, 10 aa, Artificial Sequence
  Peptide Spacer
GPGGYGPGQQ

SEQ ID NO: 17 - protein, 13 aa, Artificial Sequence
  Skin-Binding Peptide
TPFHSPENAPGSK

SEQ ID NO: 18 - protein, 12 aa, Artificial Sequence
  Skin care composition-resistant skin-binding peptide
TMGFTAPRFPHY

SEQ ID NO: 19 - protein, 12 aa, Artificial Sequence
  Skin care composition-resistant skin-binding peptide
SVSVGMKPSPRP

SEQ ID NO: 20 - protein, 15 aa, Artificial Sequence
  Skin care composition-resistant skin-binding peptide
QLSYHAYPQANHHAP

SEQ ID NO: 21 - protein, 14 aa, Artificial Sequence
  Skin care composition-resistant skin-binding peptide
SGCHLVYDNGFCDH

SEQ ID NO: 22 - protein, 13 aa, Artificial Sequence
  Skin care composition-resistant skin-binding peptide
KTMGFTAPRFPHY

SEQ ID NO: 23 - protein, 16 aa, Artificial Sequence
  Synthetic peptides
TPFHSPENAPGSGGGS

SEQ ID NO: 24 - protein, 17 aa, Artificial Sequence
  Synthetic peptides
TPFHSPENAPGSGGGSS

SEQ ID NO: 25 - protein, 15 aa, Artificial Sequence
  Synthetic peptides
TPFHSPENAPGSGGG

SEQ ID NO: 26 - protein, 7 aa, Artificial Sequence
  Synthetic peptides
FTQSLPR

SEQ ID NO: 27 - protein, 7 aa, Artificial Sequence
  Synthetic peptides
QATFMYN

SEQ ID NO: 28 - protein, 11 aa, Artificial Sequence
  Synthetic peptides
VLTSQLPNHSM

SEQ ID NO: 29 - protein, 7 aa, Artificial Sequence
  Synthetic peptides
HSTAYLT

SEQ ID NO: 30 - protein, 12 aa, Artificial Sequence
  Synthetic peptides
APQQRPMKTFNT

SEQ ID NO: 31 - protein, 12 aa, Artificial Sequence
  Synthetic peptides
APQQRPMKTVQY

SEQ ID NO: 32 - protein, 7 aa, Artificial Sequence
  Synthetic peptides
PPWLDLL

SEQ ID NO: 33 - protein, 7 aa, Artificial Sequence
  Synthetic peptides
PPWTFPL

SEQ ID NO: 34 - protein, 7 aa, Artificial Sequence
  Synthetic peptides
SVTHLTS

SEQ ID NO: 35 - protein, 7 aa, Artificial Sequence
  Synthetic peptides
VITRLTS

SEQ ID NO: 36 - protein, 12 aa, Artificial Sequence
  Synthetic peptides
DLKPPLLALSKV

SEQ ID NO: 37 - protein, 12 aa, Artificial Sequence
  Synthetic peptides
SHPSGALQEGTF

SEQ ID NO: 38 - protein, 12 aa, Artificial Sequence
  Synthetic peptides
FPLTSKPSGACT

SEQ ID NO: 39 - protein, 12 aa, Artificial Sequence
  Synthetic peptides
DLKPPLLALSKV

SEQ ID NO: 40 - protein, 7 aa, Artificial Sequence
  Synthetic peptides
PLLALHS

SEQ ID NO: 41 - protein, 7 aa, Artificial Sequence
  Synthetic peptides
VPISTQI

SEQ ID NO: 42 - protein, 12 aa, Artificial Sequence
  Synthetic peptides
YAKQHYPISTFK

SEQ ID NO: 43 - protein, 7 aa, Artificial Sequence
  Synthetic peptides
HSTAYLT

SEQ ID NO: 44 - protein, 12 aa, Artificial Sequence
  Synthetic peptides
STAYLVAMSAAP

SEQ ID NO: 45 - protein, 12 aa, Artificial Sequence
  Synthetic peptides
NLQHSVGTSPVW

SEQ ID NO: 46 - protein, 12 aa, Artificial Sequence
  Synthetic peptides
NQAASITKRVPY

SEQ ID NO: 47 - protein, 14 aa, Artificial Sequence
  Synthetic peptides
ASCPSASHADPCAH

SEQ ID NO: 48 - protein, 14 aa, Artificial Sequence
  Synthetic peptides
NLCDSARDSPRCKV

SEQ ID NO: 49 - protein, 12 aa, Artificial Sequence
  Synthetic peptides
NHSNWKTAADFL

SEQ ID NO: 50 - protein, 12 aa, Artificial Sequence
  Synthetic peptides
GSSTVGRPLSYE

SEQ ID NO: 51 - protein, 12 aa, Artificial Sequence
  Synthetic peptides
SDTISRLHVSMT

SEQ ID NO: 52 - protein, 12 aa, Artificial Sequence
  Synthetic peptides
SPLTVPYERKLL

SEQ ID NO: 53 - protein, 12 aa, Artificial Sequence
  Synthetic peptides
SPYPSWSTPAGR

SEQ ID NO: 54 - protein, 12 aa, Artificial Sequence
  Synthetic peptides
VQPITNTRYEGG

SEQ ID NO: 55 - protein, 12 aa, Artificial Sequence
  Synthetic peptides
WPMHPEKGSRWS

SEQ ID NO: 56 - protein, 14 aa, Artificial Sequence
  Synthetic peptides
DACSGNGHPNNCDR

SEQ ID NO: 57 - protein, 14 aa, Artificial Sequence
  Synthetic peptides
DHCLGRQLQPVCYP

SEQ ID NO: 58 - protein, 14 aa, Artificial Sequence
  Synthetic peptides
DWCDTIIPGRTCHG

(19) United States
(12) Patent Application Publication
(10) Pub. No.: US 2009/0142283 A1
(43) Pub. Date: Jun. 4, 2009

(54) PEPTIDE-BASED ANTIDANDRUFF REAGENTS

(75) Inventors: John P. O'Brien, Oxford, PA (US); Hong Wang, Kennett Square, PA (US); Antoinette E. Wilkins, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, WILMINGTON, DE (US)

(21) Appl. No.: 12/273,753

(22) Filed: Nov. 19, 2008

Related U.S. Application Data
(60) Provisional application No. 60/991,257, filed on Nov. 30, 2007.

(51) Int. Cl. A61K 8/64; C07K 14/00; A61K 38/16

(52) U.S. Cl. 424/59; 530/300; 8/405; 514/8

(57) ABSTRACT

Peptide-based antidandruff reagents, formed by coupling a skin-binding peptide with an antidandruff agent, are described. The skin-binding peptide part of the peptide-based antidandruff reagent binds strongly to the skin, thus keeping the antidandruff agent coupled to the skin for a long lasting effect. Hair care compositions comprising the peptide-based antidandruff reagents are also provided as well as methods for treating or preventing dandruff.

This application claims the benefit of U.S. Provisional Pat. App. No. 60/991,257, filed Nov. 30, 2007.

### FIELD OF THE INVENTION

The invention relates to the field of personal care products. More specifically, the invention relates to peptide-based antidandruff reagents formed by coupling a skin-binding peptide with an antidandruff agent.

### BACKGROUND OF THE INVENTION

Dandruff is a chronic scalp condition that causes scaling and flaking of the skin. The causes of dandruff are not entirely known. Currently, a scalp specific fungus, Malassezia globosa, is believed the likely responsible agent (Dawson, Thomas L., J. Investig. Dermatol. Symp. Proc. (2007), 12:15-19). This fungus metabolizes triglycerides present in sebum by the expression of lipase, resulting in a lipid byproduct oleic acid [“OA”]. Penetration by OA of the top layer of the epidermis, the stratum corneum, results in an inflammatory response in susceptible persons which disturbs homeostasis and results in erratic cleavage of stratum corneum cells. The primary treatment for dandruff is the topical application of antifungal agents that reduce the level of Malassezia globosa on the scalp. Typically, the antifungal agent is applied to the scalp as a component of a shampoo or other hair care composition. Examples of antidandruff compositions are described in U.S. Pat. App. Pub. No. 2004/0202636 to Kaczvinsky et al., U.S. Pat. No. 6,410,5934 to De Mesanstourne et al., and U.S. Pat. App. Pub. No. 2002/0172648 to Hehner et al. However, the antidandruff agents are in contact with the scalp for a short period of time, necessitating long, repeated use of the hair care composition. A long-lasting, durable dandruff treatment would represent an advance in the art.

In order to improve the durability of hair and skin care products, peptide-based hair conditioners, hair colorants, and other benefit agents have been developed (Huang et al., U.S. Pat. No. 7,220,405, and U.S. Pat. App. Pub. No. 2005/0226839). The peptide-based benefit agents are prepared by coupling a specific peptide sequence that has a high binding affinity to hair or skin with a benefit agent, such as a conditioner or colorant. The peptide portion binds to the hair or skin, thereby strongly attaching the benefit agent to the body surface. Additionally, U.S. Pat. No. 6,232,287 to Ruoslahti et al. describes the use of molecules that selectively home to various organs and tissues, such as skin, to deliver a therapeutic agent.

Peptides having a binding affinity to hair and skin have been identified using phage display screening techniques (Huang et al., supra; Estell et al. Int'l App. Pub. No. 0179479; Murray et al., U.S. Pat. App. Pub. No. 2002/0098524; Janssen et al., U.S. Pat. App. Pub. No. 2003/0152976; and Janssen et al., Int'l. App. Pub. No. 04048399). Additionally, empirically-generated hair- and skin-binding peptides that are based on positively charged amino acids have been reported (Rothe et al., Int'l App. Pub. No. 2004/000257).

In view of the above, a need exists for antidandruff agents that provide improved durability for long lasting effects and are easy and inexpensive to prepare. The stated need has been addressed by designing peptide-based antidandruff reagents that provide a long lasting effect by coupling skin-binding peptides, which bind to skin with high affinity, to antidandruff agents.

### SUMMARY OF THE INVENTION

Described herein are peptide-based antidandruff reagents formed by coupling at least one skin-binding peptide with at least one antidandruff agent. Described herein is a peptide-based antidandruff reagent having the general structure:

(SBPₘ)ₙ−(ADA)_{y}, wherein
a) SBP is a skin-binding peptide;
b) ADA is an antidandruff agent;
c) m ranges from 1 to about 100;
d) n ranges from 1 to about 100; and
e) y ranges from 1 to about 100.

Also described herein is a peptide-based antidandruff reagent having the general structure:

[(SBP)ₓ−Sₘ]ₙ−(ADA)_{y}, wherein
a) SBP is a skin-binding peptide;
b) ADA is an antidandruff agent;
c) S is a spacer;
d) x ranges from 1 to about 10;
e) m ranges from 1 to about 100;
f) n ranges from 1 to about 100; and
g) y ranges from 1 to about 100.

Also described herein are hair care compositions comprising an effective amount of a peptide-based antidandruff reagent.

Also described herein are methods for treating or preventing dandruff comprising applying the hair care composition described herein to the scalp. One of the methods described comprises the steps of:
a) providing a hair care composition comprising a peptide-based antidandruff reagent selected from the group consisting of:

(SBPₘ)ₙ−(ADA)_{y}; and i)

[(SBP)ₓ−Sₘ]ₙ−(ADA)_{y} ii)
wherein
   1) SBP is a skin-binding peptide;
   2) ADA is an antidandruff agent;
   3) n ranges from 1 to about 100;
   4) S is a spacer;
   5) m ranges from 1 to about 100;
   6) x ranges from 1 to about 10; and
   7) y ranges from 1 to about 100;
and wherein the skin-binding peptide is selected by a method comprising the steps of:
   A) providing a combinatorial library of DNA associated peptides;
   B) contacting the library of (A) with a skin sample to form a reaction solution comprising DNA associated peptide-skin complexes;
   C) isolating the DNA associated peptide-skin complexes of (B);
   D) amplifying the DNA encoding the peptide portion of the DNA associated peptide-skin complexes of (C); and
   E) sequencing the amplified DNA of (d) encoding a skin-binding peptide, wherein the skin-binding peptide is identified; and
b) applying the hair care composition of (a) to the scalp.

### SEQUENCE DESCRIPTIONS

The variations in the inventions recited in the claims can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-12 and 17-58 are the amino acid sequences of skin-binding peptides.

SEQ ID NO:13 is the amino acid sequence of the protease Caspase 3 cleavage site.

SEQ ID NOs:14-16 are the amino acid sequences of peptide spacers.

SEQ ID NOs:18-22 and 45-58 are the amino acid sequences of skin care composition-resistant skin-binding peptides.

### DETAILED DESCRIPTION OF THE INVENTION

Described herein are peptide-based antidandruff reagents formed by coupling at least one skin-binding peptide to at least one antidandruff agent, either directly or through a spacer. The peptide-based antidandruff reagents may be used in hair care compositions to treat or prevent dandruff. The peptide-based antidandruff reagents remain attached to the skin, through the affinity of the skin-binding peptide, thus providing a durable, long lasting effect. Due to the strong attachment of the peptide-based antidandruff reagents to the scalp, it may be possible to use lower concentrations of the reagents compared to conventional antidandruff agents.

The following definitions are used herein and should be referred to for interpretation of the claims and the specification.

As used herein, the indefinite articles “a” and “an” preceding an element or component of the invention are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof.

As used herein, the term “about” refers to modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein, the term “invention” or “present invention” is a non-limiting term and is not intended to refer to any single variation of the inventions recited in the claims and described herein.

As used herein, the term “antidandruff agent” refers to any chemical that is effective in the treatment of dandruff and/or the symptoms associated therewith.

As used herein, the term “peptide” refers to two or more amino acids joined to each other by peptide bonds or modified peptide bonds.

As used herein, the term “skin-binding peptide” refers to peptide sequences that bind with high affinity to skin. The skin-binding peptides of the invention are from about 7 amino acids to about 60 amino acids, more preferably, from about 7 amino acids to about 35 amino acids, more preferably about 7 to about 30 amino acids, and most preferably from about 7 to about 25 amino acids in length.

As used herein, the term “DNA associated peptide” refers to a peptide having associated with it an identifying nucleic acid component. Typically, the DNA associated peptide is produced as a result of a display system such as phage display. In this system, peptides are displayed on the surface of the phage while the DNA encoding the peptides is contained within the attached glycoprotein coat of the phage. The association of the coding DNA within the phage may be used to facilitate the amplification of the coding region for the identification of the peptide.

As used herein, the term “DNA associated peptide-skin complex” refers to a complex between skin and a DNA associated peptide wherein the peptide is bound to the skin via a binding site on the peptide.

As used herein, the term “skin” refers to human skin, or substitutes for human skin, such as pig skin, VITRO-SKIN® and EPIDERM™. Skin as a body surface will generally comprise a layer of epithelial cells and may additionally comprise a layer of endothelial cells.

As used herein, the term “skin surface” will mean the surface of skin that may serve as a substrate for the binding of a skin-binding peptide and/or a peptide-based antidandruff reagent.

As used herein, the terms “coupling” and “coupled” n refer to any chemical association and includes both covalent and non-covalent interactions. The term “coupling” or “coupled” may refer to a non-covalent interaction or to a covalent interaction.

As used herein, the term “stringency” as it is applied to the selection of the skin-binding peptides of the present invention, refers to the concentration of the eluting agent (usually a detergent) used to elute peptides from the skin. Higher concentrations of the eluting agent provide more stringent conditions.

As used herein, the term “MB₅₀” refers to the concentration of the binding peptide that gives a signal that is 50% of the maximum signal obtained in an ELISA-based binding assay (see Example 3 of U.S. Pat. App. Pub. 2005/022683, incorporated herein by reference). The MB₅₀ provides an indication of the strength of the binding interaction or affinity of the components of the complex. The lower the value of MB₅₀, the stronger the interaction of the peptide with its corresponding substrate.

As used herein, the terms “binding affinity” or “affinity” refer to the strength of interaction a binding peptide has with its respective substrate. The binding affinity may be reported as a MB₅₀ value, determined in an ELISA-based binding assay, or as a K_{D} (equilibrium dissociation constant) value, deduced using surface plasmon resonance (SPR).

As used herein, the term “strong affinity” refers to a binding affinity, as measured as an MB₅₀ or K_{D} value, of 10⁻⁴ M, preferably 10⁻⁵ M or less, preferably less than 10⁻⁶ M, more preferably less than 10⁻⁷ M, more preferably less than 10⁻⁸ M, even more preferably less than 10⁻⁹ M, and most preferably less than 10⁻¹⁰ M. The lower the value of MB₅₀ or K_{D}, the stronger affinity of the peptide interacting with its corresponding substrate.

As used herein, the term “amino acid” refers to the basic chemical structural unit of a protein or polypeptide and amino acid [“aa”] abbreviations used herein are the following:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| (or as defined herein) | | |

As used herein, “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

As used herein, “synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

As used herein, “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “expression” refers to the transcription and stable accumulation of sense (mRNA) or antisense RNA derived from the nucleic acid fragment of the invention. Expression may also refer to translation of mRNA into a polypeptide.

As used herein, the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

As used herein, the term “host cell” refers to a cell which has been transformed or transfected, or is capable of transformation or transfection by an exogenous polynucleotide sequence.

As used herein, the terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

As used herein, the term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

As used herein, “PCR” or “polymerase chain reaction” is a technique used for the amplification of specific DNA segments (U.S. Pat. Nos. 4,683,195 and 4,800,159).

As used herein, the term “peptide-based” refers to an interfacial material comprised of a compound pertaining to or having the nature or the composition of the peptide class. Interfacial refers to the quality of the peptide-based material described herein as connecting one material to another.

The following abbreviates are used herein:
“SBP” means skin-binding peptide.
“ADA” means antidandruff agent.
“S” means spacer

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Skin-Binding Peptides

Skin-binding peptides (SBP), as defined herein, are peptide sequences that bind with high affinity to skin. The skin-binding peptides of the invention are from about 7 amino acids to about 60 amino acids, more preferably, from about 7 amino acids to about 35 amino acids, more preferably from about 7 to about 30 amino acids in length, and most preferably from about 7 to about 25 amino acids in length. Suitable skin-binding peptides may be selected using methods that are well known in the art or may be generated empirically.

The skin-binding peptides may be generated randomly and then selected against a specific skin sample based upon their binding affinity for skin, as described by Huang et al. in U.S. Pat. No. 7,220,405. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500); ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate such biological peptide libraries are also described in Dani, M., J. of Receptor & Signal Transduction Res., 21(4):447-468 (2001), Sidhu et al., Methods in Enzymology 328:333-363 (2000), Kay et al., Combinatorial Chemistry & High Throughput Screening, Vol. 8:545-551 (2005), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England Biolabs (Beverly, Mass.).

A preferred method to randomly generate peptides is by phage display. Since its introduction in 1985, phage display has been widely used to discover a variety of ligands including peptides, proteins and small molecules for drug targets (Dixit, J. of Sci. & Ind. Research, 57:173-183 (1998)). The applications have expanded to other areas such as studying protein folding, novel catalytic activities, DNA-binding proteins with novel specificities, and novel peptide-based biomaterial scaffolds for tissue engineering (Hoess, Chem. Rev. 101:3205-3218 (2001) and Holmes, Trends Biotechnol. 20:16-21 (2002)). Whaley et al. (Nature 405:665-668 (2000)) disclose the use of phage display screening to identify peptide sequences that can bind specifically to different crystallographic forms of inorganic semiconductor substrates.

A modified screening method that comprises contacting a peptide library with an anti-target to remove peptides that bind to the anti-target, then contacting the non-binding peptides with the target has been described (Estell et al. Int'l App. Pub. No. 01/79479, Murray et al. U.S. Pat. App. Pub. No. 2002/0098524, and Janssen et al. U.S. Pat. App. Pub. No. 2003/0152976). Using that method, a peptide binds to hair and not to skin and a peptide that binds to skin and not hair were identified. Using the same method, Janssen et al. (Int'l. App. Pub. No. 04/048399) identified other skin-binding and hair-binding peptides, as well as several other binding motifs.

Phage display is a selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. As used herein, “biopanning” may be used to describe any selection procedure (phage display, ribosome display, mRNA-display, etc.) where a library of displayed peptides a library of displayed peptides is panned against a specified target material (e.g. hair). In its simplest form, phage display biopanning is carried out by incubating the pool of phage-displayed variants with a target of surface interest (the target material is often immobilized on a plate or bead), washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

The skin-binding peptides may be identified using the following process. A suitable library of phage-peptides is generated using the methods described above or the library is purchased from a commercial supplier. After the library of phage-peptides has been generated, the library is contacted with an appropriate amount of skin sample to form a reaction solution. Human skin samples may be obtained from cadavers or in vitro human skin cultures. Additionally, pig skin, VITRO-SKIN® (available from IMS inc., Milford, Conn.) and EPIDERM™ (available from Mattek corp., Ashland, Mass.) may be used as substitutes for human skin. The library of phage-peptides is dissolved in a suitable solution for contacting the skin substrate. The test substrate may be suspended in the solution or may be immobilized on a plate or bead. A preferred solution is a buffered aqueous saline solution containing a surfactant. A suitable solution is Tris-buffered saline (TBS) with 0.05 to 0.5% TWEEN® 20. The solution may additionally be agitated by any means in order to increase the mass transfer rate of the peptides to the substrate, thereby shortening the time required to attain maximum binding.

Upon contact, a number of the randomly generated phage-peptides will bind to the substrate to form a phage-peptide-substrate complex. Unbound phage-peptide may be removed by washing. After all unbound material is removed, phage-peptides having varying degrees of binding affinities for the substrate may be fractionated by selected washings in buffers having varying stringencies. Increasing the stringency of the buffer used increases the required strength of the bond between the phage-peptide and substrate in the phage-peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred. It will be appreciated that phage-peptides having increasing binding affinities for the substrate may be eluted by repeating the selection process using buffers with increasing stringencies. The eluted phage-peptides can be identified and sequenced by any means known in the art.

For example, the eluted DNA associated peptides and the remaining bound DNA associated peptides may be amplified by infecting/transfecting a bacterial host cell, such as E. coli ER2738, as described by Huang et al. (U.S. Pat. No. 7,220,405). The infected host cells are grown in a suitable growth medium, such as LB (Luria-Bertani) medium, and this culture is spread onto agar, containing a suitable growth medium, such as LB medium with IPTG (isopropyl β-D-thiogalactopyranoside) and S-Gal™ (3,4-cyclohexenoesculetin-β-D-galactopyranoside). After growth, the plaques are picked for DNA isolation and sequencing to identify the skin-binding peptide sequences. Alternatively, the eluted DNA associated peptides and the remaining bound DNA associated peptides may be amplified using a nucleic acid amplification method, such as the polymerase chain reaction (PCR), to amplify the DNA comprising the peptide coding region. In that approach, PCR is carried out on the DNA encoding the eluted DNA associated peptides and/or the remaining bound DNA associated peptides using the appropriate primers, as described by Janssen et al. in U.S. Pat. App. Pub. No. 2003/0152976, which is incorporated herein by reference.

The eluted DNA associated peptides and the remaining bound DNA associated peptides may be amplified by infecting a bacterial host cell as described above, the amplified DNA associated peptides are contacted with a fresh skin sample, and the entire process described above is repeated one or more times to obtain a population that is enriched in skin-binding DNA associated peptides. After the desired number of biopanning cycles, the amplified DNA associated peptide sequences are determined using standard DNA sequencing techniques that are well known in the art to identify the skin-binding peptide sequences. Skin-binding peptide sequences identified using this method include, but are not limited to SEQ ID NO:1, 7-12, 17, and 23-44.

Skin-binding peptides that are resistant to skin care compositions, as described by Wang et al. in U.S. patent application Ser. No. 11/359162 (published as U.S. 2006/0199206), may also be used in the peptide-based antidandruff reagents of the invention. Examples of skin care composition-resistant skin-binding peptides include, but are not limited to, the peptide sequences given as SEQ ID NOs:18-22 and 45-58.

The skin-binding peptide may be selected from the group consisting of SEQ ID NOs:1, 2, 3, 4, 5, 6, 7, 8, 9, 10, 11, 12, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, and 58.

Additionally, skin-binding peptides that are resistant to shampoo and other hair care compositions, such as conditioners, may be identified using variations of the methods described by O'Brien et al. in U.S. Pat. App. Pub. No. 2006/0073111, Wang et al. in U.S. Pat. App. Pub. No. 2007/0196305, and Wang et al. in U.S. Pat. App. Pub. No. 2006/0199206 for identifying shampoo resistant hair-binding peptides, hair conditioner-resistant hair-binding peptides, and skin care composition-resistant skin-binding peptides, respectively. Briefly, the DNA associated peptide-skin complex is contacted with the desired composition (e.g. a shampoo or hair conditioner) at least one time in the biopanning process described above. For example, the phage peptide library may be dissolved in the hair care composition which is then contacted with the skin sample. Alternatively, the DNA associated peptide-skin complex, formed by contacting the skin sample with the phage display library, may be subsequently contacted with a hair care composition. Additionally, any combination of these hair care composition-contacting methods may be used.

Skin-binding peptide sequences may also be determined using the method described by Lowe, D. in U.S. Pat. App. Pub. No. 2006/0286047. The Lowe method provides a means for determining the sequence of a peptide binding motif having affinity for a particular substrate, for example skin. First, a population of binding peptides for the substrate of interest is identified by biopanning using a combinatorial method, such as phage display. Rather than using many rounds of biopanning to identify specific binding peptide sequences and then using standard pattern recognition techniques to identify binding motifs, as is conventionally done in the art, the method requires only a few rounds of biopanning. The sequences in the population of binding peptides, which are generated by biopanning, are analyzed by identifying subsequences of 2, 3, 4, and 5 amino acid residues that occur more frequently than expected by random chance. The identified subsequences are then matched head to tail to give peptide motifs with substrate binding properties. This procedure may be repeated many times to generate long peptide sequences.

Alternatively, skin-binding peptide sequences may be generated empirically by designing peptides that comprise positively charged amino acids, which can bind to skin via electrostatic interaction, as described by Rothe et al. in Int'l. App. Pub. No. 2004/000257. The empirically generated skin-binding peptides have between about 7 amino acids to about 60 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically generated skin-binding peptides include, but are not limited to, SEQ ID NOs: 2, 3, 4, 5, and 6.

The skin-binding peptide may further comprise at least one cysteine or lysine residue on at least one of the C-terminal end or the N-terminal end of the skin-binding peptide sequence to facilitate coupling with the antidandruff agent, as described below. Examples of skin-binding peptides having a lysine residue on the C-terminal end of the binding sequence are given as SEQ ID NOs: 17 and 42. Additionally, the skin-binding peptide may further comprise at least one proline or aspartic acid residue on at least one of the C-terminal end or the N-terminal end of the skin-binding peptide sequence. The terminal aspartic acid (D) or proline (P) residues may result from the use of acid-labile DP cleavage sites in the biological production of the peptides. Examples of various skin-binding peptides are provided below in Table A.

TABLE A
Examples of Skin-Binding Peptides

| | | | |
|---|---|---|---|
| Hair and | KRGRHKRPKRHK | 2 | US 2007-0065387 |
| skin | | | US 2007-0110686 |
| (Empirical) | | | US 2007-0067924 |
| Hair and | RLLRLLR | 3 | US 2007-0065387 |
| skin | | | US 2007-0110686 |
| (Empirical) | | | |
| Hair and | HKPRGGRKKALH | 4 | US 2007-0065387 |
| skin | | | US 2007-0110686 |
| (Empirical) | | | |
| Hair and | KPRPPHGKKHRPKHRPKK | 5 | US 2007-0065387 |
| skin | | | US 2007-0110686 |
| (Empirical) | | | |
| Hair and | RGRPKKGHGKRPGHRARK | 6 | US 2007-0065387 |
| skin | | | US 2007-0110686 |
| (Empirical) | | | |
| Skin | TPFHSPENAPGS | 1 | US 11/877,692 |
| | | | US 2005-0249682 |
| Skin | TPFHSPENAPGSK | 17 | US 2007-0110686 |
| Skin | TPFHSPENAPGSGGGS | 23 | US 2007-0110686 |
| Skin | TPFHSPENAPGSGGGSS | 24 | US 2007-0110686 |
| Skin | TPFHSPENAPGSGGG | 25 | US 2007-0110686 |
| Skin | FTQSLPR | 26 | US 11/877,692 |
| | | | US 2005-0249682 |
| Skin | KQATFPPNPTAY | 7 | US 11/877,692 |
| | | | US 2005-0249682 |
| | | | WO2004048399 |
| Skin | HGHMVSTSQLSI | 8 | US 11/877,692 |
| | | | US 2005-0249682 |
| | | | WO2004048399 |
| Skin | LSPSRMK | 9 | US 11/877,692 |
| | | | US 2005-0249682 |
| | | | WO2004048399 |
| Skin | LPIPRMK | 10 | US 2005-0249682 |
| | | | WO2004048399 |
| Skin | HQRPYLT | 11 | US 2005-0249682 |
| | | | WO2004048399 |
| Skin | FPPLLRL | 12 | US 2005-0249682 |
| | | | WO2004048399 |
| Skin | QATFMYN | 27 | WO2004048399 |
| Skin | VLTSQLPNHSM | 28 | WO2004048399 |
| Skin | HSTAYLT | 29 | WO2004048399 |
| Skin | APQQRPMKTFNT | 30 | WO2004048399 |
| Skin | APQQRPMKTVQY | 31 | WO2004048399 |
| Skin | PPWLDLL | 32 | WO2004048399 |
| Skin | PPWTFPL | 33 | WO2004048399 |
| Skin | SVTHLTS | 34 | WO2004048399 |
| Skin | VITRLTS | 35 | WO2004048399 |
| Skin | DLKPPLLALSKV | 36 | WO2004048399 |
| Skin | SHPSGALQEGTF | 37 | WO2004048399 |
| Skin | FPLTSKPSGACT | 38 | WO2004048399 |
| Skin | DLKPPLLALSKV | 39 | WO2004048399 |
| Skin | PLLALHS | 40 | WO2004048399 |
| Skin | VPISTQI | 41 | WO2004048399 |
| Skin | YAKQHYPISTFK | 42 | WO2004048399 |
| Skin | HSTAYLT | 43 | WO2004048399 |
| Skin | STAYLVAMSAAP | 44 | WO2004048399 |
| Skin (Body | SVSVGMKPSPRP | 19 | US 11/877,692 |
| Wash | | | US 2006-0199206 |
| Resistant) | | | |
| Skin (Body | TMGFTAPRFPHY | 18 | US 11/877,692 |
| Wash | | | US 2006-0199206 |
| Resistant) | | | |
| Skin (Body | KTMGFTAPRFPHY | 22 | US 11/877,692 |
| Wash | | | US 2006-0199206 |
| Resistant) | | | |
| Skin (Body | NLQHSVGTSPVW | 45 | US 11/877,692 |
| Wash | | | US 2006-0199206 |
| Resistant) | | | |
| Skin (Body | QLSYHAYPQANHHAP | 20 | US 11/877,692 |
| Wash | | | US 2006-0199206 |
| Resistant) | | | |
| Skin (Body | NQAASITKRVPY | 46 | US 2006-0199206 |
| Wash | | | |
| Resistant) | | | |
| Skin (Body | SGCHLVYDNGFCDH | 21 | US 11/877,692 |
| Wash | | | US 2006-0199206 |
| Resistant) | | | |
| Skin (Body | ASCPSASHADPCAH | 47 | US 11/877,692 |
| Wash | | | US 2006-0199206 |
| Resistant) | | | |
| Skin (Body | NLCDSARDSPRCKV | 48 | US 11/877,692 |
| Wash | | | US 2006-0199206 |
| Resistant) | | | |
| Skin (Body | NHSNWKTAADFL | 49 | US 11/877,692 |
| Wash | | | US 2006-0199206 |
| Resistant) | | | |
| Skin (Body | GSSTVGRPLSYE | 50 | US 2006-0199206 |
| Wash | | | |
| Resistant) | | | |
| Skin (Body | SDTISRLHVSMT | 51 | US 11/877,692 |
| Wash | | | US 2006-0199206 |
| Resistant) | | | |
| Skin (Body | SPLTVPYERKLL | 52 | US 2006-0199206 |
| Wash | | | |
| Resistant) | | | |
| Skin (Body | SPYPSWSTPAGR | 53 | US 11/877,692 |
| Wash | | | US 2006-0199206 |
| Resistant) | | | |
| Skin (Body | VQPITNTRYEGG | 54 | US 2006-0199206 |
| Wash | | | |
| Resistant) | | | |
| Skin (Body | WPMHPEKGSRWS | 55 | US 2006-0199206 |
| Wash | | | |
| Resistant) | | | |
| Skin (Body | DACSGNGHPNNCDR | 56 | US 11/877,692 |
| Wash | | | US 2006-0199206 |
| Resistant) | | | |
| Skin (Body | DHCLGRQLQPVCYP | 57 | US 2006-0199206 |
| Wash | | | |
| Resistant) | | | |
| Skin (Body | DWCDTIIPGRTCHG | 58 | US 11/877,692 |
| Wash | | | US 2006-0199206 |
| Resistant) | | | |

### Binding Affinity

The skin-binding peptides in the peptide-based antidandruff reagents described herein may exhibit a strong affinity for skin. The affinity of the peptide for the skin can be expressed in terms of the dissociation constant K_{D}. K_{D} (expressed as molar concentration) corresponds to the concentration of peptide at which the binding site on the target is half occupied, i.e. when the concentration of target with peptide bound (bound target material) equals the concentration of target with no peptide bound. The smaller the dissociation constant, the more tightly bound the peptide is; for example, a peptide with a nanomolar (nM) dissociation constant binds more tightly than a peptide with a micromolar (μM) dissociation constant. The skin-binding peptides may have a K_{D} of 10⁻⁴ M or less, preferably 10⁻⁵ M or less, more preferably 10⁻⁶ M or less, even more preferably 10⁻⁷ M or less, yet even more preferably 10⁻⁸ M or less, and most preferably 10⁻⁹ M or less.

Alternatively, one of skill in the art can also use an ELISA-based assay to calculate a relative affinity of the peptide for the target material (reported as an “MB₅₀” value; see Example 3 of U.S. Pat. App. Pub. 2005/022683, incorporated herein by reference). As used herein, the term “MB₅₀” refers to the concentration of the binding peptide that gives a signal that is 50% of the maximum signal obtained in an ELISA-based binding assay. The MB₅₀ provides an indication of the strength of the binding interaction or affinity of the components of the complex. The lower the value of MB₅₀, the stronger the interaction of the peptide with its corresponding substrate. The MB₅₀ value (reported in terms of molar concentration) for the skin-binding peptide may be 10⁻⁴ M or less, preferably 10⁻⁵ M or less, more preferably 10⁻⁶ M or less, even more preferably 10⁻⁷ M or less, and most preferably 10⁻⁸ M or less.

### Production of Skin-Binding Peptides

The skin-binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the skin-binding peptides may be produced in heterologous host cells, particularly in the cells of microbial hosts, as described by Huang et al. in U.S. Pat. No. 7,220,405; herein incorporated by reference.

Preferred heterologous host cells for expression of the skin-binding peptides are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides described herein. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the skin-binding peptides or peptide-based reagents described herein. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the peptides described herein including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence is typically employed to transform an appropriate host to permit the host to express the peptide of the present invention. Cell-free translation systems can also be employed to produce such peptides using RNAs derived from the DNA constructs of the present invention. Optionally it may be desired to produce the gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP Pub. No. 546049 and Int'l App. Pub. No. 9324631). The secretion signal DNA or facilitator may be located between the expression-controlling DNA and the instant gene or gene fragment, and in the same reading frame with the latter.

### Peptide-Based Antidandruff Reagents

The peptide-based antidandruff reagents of the present invention are formed by coupling at least one skin-binding peptide (SBP) with at least one antidandruff agent (ADA). The skin-binding peptide part of the antidandruff reagent binds strongly to the skin, thus keeping the antidandruff agent attached to the skin for a long lasting effect. Suitable skin-binding peptides include, but are not limited to, the skin binding peptides described above. It may also be desirable to link two or more skin-binding peptides together, either directly or through a spacer, to enhance the interaction with the skin. Methods to prepare these multiple skin-binding peptides and suitable spacers are described below.

Antidandruff agent, as herein defined, refers to any chemical that is effective in the treatment of dandruff and/or the symptoms associated therewith. Antidandruff agents are well known in the art (see for example, U.S. Pat. App. Pub. No. 2004/0202636 to Kaczvinsky et al. (in particular, paragraphs 0041-0053); U.S. Pat. App. Pub. No. 2003/0003070 to Eggers et al. (in particular, paragraph 007); and U.S. Pat. No. 6,284,234 to Niemiec et al. (in particular, column 13, lines 14-33), all of which are incorporated herein by reference). Typically, the antidandruff agent is an antifungal agent effective against the fungus Malassezia globosa. Suitable antidandruff agents include, but are not limited to pyridinethione salts, such as calcium, magnesium, barium, strontium, zinc, and zirconium pyridinethione salts; azoles, such as climbazole (CAS No. 38083-17-9), ketoconazole (CAS No. 65277-42-1) ,and itraconazole (CAS No. 84625-61-6); piroctone olamine (octopirox); undecylenic acid (CAS No. 112-38-9), undecylenamidopropylbetaine (AMPHORAM U®), coal tar (Neutrogena T/gel, CAS No. 8030-31-7); salisylic acid (Ionil T, CAS No. 69-72-7); selenium sulfide (Selsun Blue, CAS No. 7446-34-6) and Tea tree oil (CAS No. 68647-73-4), and mixtures thereof. A preferred pyridinethione salt is the zinc salt of 1-hydroxy-2-pyridinethione (CAS No. 13463-41-7; also known as zinc pyridinethione). These antifungal agents are generally available from commercial sources. For example, zinc pyridinethione is available from Olin Corporation (Norwalk, Conn.); octopirox is available from Hoechst AG (Frankfurt, Germany); AMPHORAM U® is available from CECA Arkema Group (France); and ketoconazole is available from Alfa Chem (Kings Point, N.Y.).

The antidandruff agent may be selected from the group consisting of a calcium pyridinethione salt, a magnesium pyridinethione salt, a barium pyridinethione salt, a strontium pyridinethione salt, a zinc pyridinethione salt, a zirconium pyridinethione salt, piroctone olamine, climbazole, ketoconazole, itraconazole, undecylenic acid, undecylenamidopropylbetaine, and combinations thereof.

Additionally, the antidandruff agent may be an antifungal peptide having activity against Malassezia globosa. Antifungal peptides are well known in the art (see for example, De Lucca et al., Rev. Iberoam. Micol. 17:116-120 (2000)). The antifungal peptide may be a naturally occurring peptide or an analog thereof, or it may be a synthetic peptide. As used herein, the term “analog” refers to a naturally occurring antifungal peptide that has been chemically modified to improve its effectiveness and/or reduce its toxic effects side effects. Synthetic antifungal peptides have been described for the treatment of dandruff (see for example, Christophers et al. U.S. Pat. No. 6,255,279, Hogenhaug, U.S. Pat. App. Pub. No. 2005/0239709, Strom et al., U.S. Pat. App. Pub. No. 2005/0187151, Hart et al., U.S. Pat. App. Pub. No. 2005/0282755, and Hogenhaug et al., U.S. Pat. App. Pub. No. 2005/0245452, all of which are incorporated herein by reference). Suitable antifungal peptides can include, but are not limited to, syringomycins, syringostatins, syringotoxins, nikkomycins, echinocandins, pneumocadins, aculeacins, mulundocadins, cecropins, α-defensins, β-defensins, novispirins, and combinations thereof. These antifungal peptides may be prepared using the methods described above for the preparation of skin-binding peptides.

The antifungal peptide may be selected from the group consisting of syringomycins, syringostatins, syringotoxins, nikkomycins, echinocandins, pneumocadins, aculeacins, mulundocadins, cecropins, α-defensins, β-defensins, novispirins, and mixtures thereof.

The peptide-based antidandruff reagents are prepared by coupling at least one specific skin-binding peptide to at least one antidandruff agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based antidandruff reagent may be prepared by mixing the peptide with the antidandruff agent and the optional spacer (if used) and allowing sufficient time for the interaction to occur. The unbound materials may be separated from the resulting peptide-based antidandruff reagent using methods known in the art, for example, liquid chromatography.

The peptide-based antidandruff reagents of the invention may also be prepared by covalently attaching at least one specific skin-binding peptide to at least one antidandruff agent, either directly or through a spacer. Any known peptide or protein conjugation chemistry may be used to form the peptide-based antidandruff reagents of the present invention. Conjugation chemistries are well-known in the art (see for example, G. T. Hermanson, Bioconiugate Techniques, 2^{nd} Ed., Academic Press, New York (2008)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based antidandruff reagent. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, isocyanate, epoxide, or aldehyde groups on the antidandruff agent for coupling to the skin-binding peptide. These modifications may be done using routine chemistry such as oxidation, reduction, phosgenation, and the like, which is well known in the art.

It may also be desirable to couple the skin-binding peptide to the antidandruff agent via a spacer. The spacer serves to separate the antidandruff agent from the peptide to ensure that the agent does not interfere with the binding of the peptide to the skin. The spacer may be any of a variety of molecules, such as alkyl chains, phenyl compounds, ethylene glycol, amides, esters and the like. Preferred spacers have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, and ethyl, propyl, hexyl, steryl, cetyl, and palmitoyl alkyl chains. The spacer may be covalently attached to the peptide and the antidandruff agent using any of the coupling chemistries described above. In order to facilitate incorporation of the spacer, a bifunctional cross-linking agent that contains a spacer and reactive groups at both ends for coupling to the peptide and the antidandruff agent may be used.

Additionally, the spacer may be a peptide comprising any amino acid and mixtures thereof. The preferred peptide spacers are comprised of the amino acids proline, lysine, glycine, alanine, and serine, and mixtures thereof. In addition, the peptide spacer may comprise a specific enzyme cleavage site, such as the protease Caspase 3 site, given as SEQ ID NO:13, which allows for the enzymatic removal of the antidandruff agent from the skin. The peptide spacer may be from 1 to about 50 amino acids, preferably from 1 to about 20 amino acids in length. Suitable peptide spacers comprise amino acid sequences including, but are not limited to, SEQ ID NOs: 14, 15, and 16. These peptide spacers may be linked to the binding peptide sequence by any method known in the art. For example, the entire binding peptide-peptide spacer diblock may be prepared using the standard peptide synthesis methods described above. In addition, the binding peptide and peptide spacer blocks may be combined using carbodiimide coupling agents (see for example, Hermanson, supra), diacid chlorides, diisocyanates and other difunctional coupling reagents that are reactive to terminal amine and/or carboxylic acid groups on the peptides. Alternatively, the entire binding peptide-peptide spacer diblock may be prepared using the recombinant DNA and molecular cloning techniques described above. The spacer may also be a combination of a peptide spacer and an organic spacer molecule, which may be prepared using the methods described above.

When the antidandruff agent is an antifungal peptide, the skin-binding peptide may be coupled to the antifungal peptide, with or without a spacer, using the methods described above. For example, the entire skin binding peptide-antifungal peptide diblock or the skin binding peptide-peptide spacer-antifungal peptide triblock may be prepared using the standard peptide synthesis methods described above. In addition, the skin binding peptide, the optional peptide spacer, and the antifungal peptide blocks may be combined using coupling agents, as described above. Alternatively, the entire skin binding peptide-optional peptide spacer-antifungal peptide diblock or triblock may be prepared using the recombinant DNA and molecular cloning techniques described above.

It may also be desirable to have multiple skin-binding peptides coupled to the antidandruff agent to enhance the interaction between the peptide-based antidandruff reagent and the skin. Either multiple copies of the same skin-binding peptide or a combination of different skin-binding peptides may be used. Typically, 1 to about 100 skin-binding peptides can be coupled to an antidandruff agent. Additionally, multiple peptide sequences may be linked together and attached to the antidandruff agent, as described above. Typically, up to about 100 skin-binding peptides may be linked together. Moreover, multiple antidandruff agents (ADA) may be coupled to the skin-binding peptide. Therefore, the peptide-based antidandruff reagents may be compositions consisting of a skin-binding peptide (SBP) and an antidandruff agent (ADA), having the general structure (SBPₘ)ₙ−(ADA)_{y}, where m, n and y independently range from 1 to about 100, preferably from 1 to about 10.

The peptide-based antidandruff reagents may contain a spacer (S) separating the skin-binding peptide from the antidandruff agent, as described above. Multiple copies of the skin-binding peptide may be coupled to a single spacer molecule. Additionally, multiple copies of the peptides may be linked together via spacers and coupled to the antidandruff agent via a spacer. Moreover, multiple antidandruff agents (ADA) may be coupled to the spacer. Upon containing a spacer, the peptide-based antidandruff reagents are compositions consisting of a skin-binding peptide, a spacer, and an antidandruff agent, having the general structure [(SBP)ₓ−Sₘ]ₙ−(ADA)_{y}, where x ranges from 1 to about 10, and m, n and y independently range from 1 to about 100, preferably from 1 to about 10. x maybe 1.

It should be understood that as used herein, SBP is a generic designation and is not meant to refer to a single skin-binding peptide sequence. Where m, n or x as used above, is greater than 1, it is well within the scope of the invention to provide for the situation where a series of skin-binding peptides of different sequences may form a part of the composition. It should also be understood that as used herein, ADA is a generic term and is not meant to refer to a single antidandruff agent. Where y as used above, is greater than 1, it is well within the scope of the invention to provide for the situation where a number of different antidandruff agents may form a part of the composition. Additionally, it should be understood that these structures do not necessarily represent a covalent bond between the peptide, the antidandruff agent, and the optional spacer. As described above, the coupling interaction between the peptide, the antidandruff agent, and the optional spacer may be either covalent or non-covalent.

### Hair Care Compositions

The peptide-based antidandruff reagents of the invention may be used in hair care compositions to treat or prevent dandruff. Hair care compositions are herein defined as compositions for the treatment of hair including, but not limited to, shampoos, conditioners, rinses, lotions, aerosols, gels, mousses, and hair dyes. The hair care compositions of the present invention comprise an effective amount of at least one peptide-based antidandruff reagent, ranging from about 0.001% to about 10%, preferably from about 0.1% to about 5%, and more preferably from about 0.5% to about 3% by weight relative to the total weight of the composition. As used here, the term “effective amount” is that amount of the peptide-based antidandruff reagent in the hair care composition necessary to achieve the desired improvement.

The hair care composition may comprise a cosmetically acceptable medium for hair care compositions, examples of which are described for example by Philippe et al. in U.S. Pat. No. 6,280,747, by Omura et al. in U.S. Pat. No. 6,139,851 and by Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, viscosifying agents, wetting agents, anionic polymers, nonionic polymers, amphoteric polymers, viscosity/foam stabilizers, opacifying/pearlizing agents, sequestering agents, stabilizing agents, hair conditioning agents, humectants, anti-static agents, anti-freezing agents, buffering agents, dyes, and pigments. These adjuvants are well known in the field of cosmetics and are described in many publications, for example see Harry's Book of Cosmeticology, 8^{th} edition, Martin Rieger, ed., Chemical Publishing, New York (2000).

The peptide-based antidandruff reagent may be used in a shampoo. Suitable shampoo compositions are well known in the art. For example, components of shampoo compositions are described by Wells et al. in U.S. Pat. No. 6,930,078, by Patel et al. in U.S. Pat. No. 5,747,436 and by Niemiec et al. in U.S. Pat. No. 6,908,889. The hair shampoo composition can be an aqueous solution, aqueous-alcoholic solution or an oil-in-water (O/W) or water in oil in water (W/O/W) emulsion. The shampoo composition of the invention contains an effective amount of peptide-based antidandruff reagent from about 0.001 % to about 10%, preferably from about 0.1% to about 5%, and more preferably from about 0.5% to about 3% by weight relative to the total weight of the composition. The balance of the shampoo composition is comprised of the fluid vehicle, surfactant, and other additives. Typically, the fluid vehicle comprises water and other solvents which can include, without limitation, mineral oils and fatty alcohols.

Surfactants are the primary components in shampoo compositions. The amount of primary surfactant is generally in the range of between about 10% and 20% as based on the final weight of the composition, more typically from about 8 to about 18%. A secondary surfactant may also be present, generally in the range of about 0 to about 6%. The surfactants in the shampoo composition according to the invention may include one or more, or a combination thereof of anionic, nonionic, amphoteric or cationic surfactants. Examples of anionic surfactants include, but are not limited to, soaps, alkyl and alkyl ether sulfates, and alpha-olefin sulfonates. The preferred anionic surfactants are lauryl (ammonium, sodium, triethanolamine and diethanolamine and laureth (sodium and ammonium)) sulfates. Secondary anionic surfactants include, but are not limited to, sulfosuccinates, linear alkylbenzene sulfonates, N-acyl methyltaurates, N-acyl sarcosinates, acyl isothionates, N-acyl polypeptide condensates, polyalkoxylated ether glycolates, monoglyceride sulfates, fatty glycerol ether sulfonates. Examples of nonionic surfactants include, but are not limited to, fatty alkanolamides, amine oxides, polymeric ethers, polysorbate 20, PEG-80 sorbitan, and nonoxynols. Examples of amphoteric surfactants include, but are not limited to, betaines, alkyl-substituted amino acids (sodium lauraminopropionate and sodium lauriminopropionate).

The shampoo composition according to the invention may also comprise viscosity and foam stabilizers, the amount of, generally in the range of about 1.5 to about 5% based on the final weight of the composition. Specific examples of viscosity/foam stabilizers include, but are not limited to, alkanolamides (such as Cocamide MEA).

Additionally, the shampoo composition may contain minor proportions of one or more conventional cosmetic or dermatological additives or adjuvants, provided that they do not interfere with the mildness, performance or aesthetic characteristics desired in the final products. The total concentration of added ingredients usually is less than 5%, preferably less than 3%, by weight of the total composition. Such minor components include but are not limited to, opacifying/pearlizing agents, such as stearic acid derivatives (e.g., ethylene glycol monostearate or ethylene glycol distearate); solvents; sequestering agents, such as disodium ethylene diaminetetraacetic acid (EDTA) and its salts, citric acid, or polyphosphates; stabilizing agents; viscosifying agents, such as salts (e.g, sodium chloride or ammonium chloride) for anionic formulations; PEG-120 methyl glucose dioleate and PEG-150 pentaerythrityl tetrastearate for anionic/nonionic formulations; hair conditioning agents, such as the cationic polymers polyquaternium 10 (Ucare Polymers), cationic guar (Jacquar C-261N), polyquaternium-7 (Merquat Polymers) and silicones such as dimethicone and aminodimethicone; humectants; anti-static agents; anti-freezing agents, buffering agents; antioxidants, such as BHT, BHA and tocopherol; UV absorbers, such as benzophenone; preservatives, such as parabens; fragrances; and dyes or pigments. These adjuvants are well known in the field of cosmetics and are described in many publications, for example see Harry's Book of Cosmeticology, supra.

The final essential component in the shampoo composition is water, which provides an aqueous medium that constitutes the balance of the shampoo composition. Generally, the proportion of water ranges from about 53% to about 95%, preferably, 68% to about 92%, and most preferably about 80% to about 87%, by weight of the resultant shampoo composition.

The shampoo compositions of the present invention may be prepared using conventional formulation and mixing techniques. Where melting or dissolution of solid surfactants or wax components is required these can be added to a premix of the surfactants, or some portion of the surfactants, mixed and heated to melt the solid components, e.g., about 72° C. This mixture can then optionally be processed through a high shear mill and cooled, and then the remaining components are mixed in. The compositions typically have a final viscosity of from about 2,000 to about 20,000 cps (centipoise). The viscosity of the composition may be adjusted by conventional techniques including addition of sodium chloride or ammonium xylenesulfonate as needed.

### Methods for Treating or Preventing Dandruff

Also described herein are methods for treating or preventing dandruff comprising applying a hair care composition comprising at least one peptide-based antidandruff reagent, as described above, to the scalp. The hair care composition may be rinsed from the scalp or left on the scalp, depending upon the type of composition used. The compositions described herein may be applied to the scalp by various means, including, but not limited to spraying, brushing, and applying by hand.

### EXAMPLES

The inventions recited in the claims are further illustrated in the following Examples. The meaning of abbreviations used is as follows: “min” means minute(s), “h” or “hr” means hour(s), “μL” means microliter(s), “mL” means milliliter(s), “L” means liter(s), “nm” means nanometer(s), “mm” means millimeter(s), “cm” means centimeter(s), “μm” means micrometer(s) or micron(s), “mM” means millimolar, “M” means molar, “mmol” means millimole(s), “μmole” means micromole(s), “g” means gram(s), “pg” means microgram(s), “mg” means milligram(s), “g” means the gravitation constant, “rpm” means revolutions per minute, “qs” means as much as suffices, “wt %” means weight percent and “MALDI mass spectrometry” means matrix-assisted, laser desorption ionization mass spectrometry.

### Example 1 (Prophetic)

### Preparation of a Peptide-Based Antidandruff Reagent

The purpose of this prophetic Example is to describe how to prepare a peptide-based antidandruff reagent by covalently coupling an antidandruff agent to a skin-binding peptide. The peptide-based antidandruff reagent is prepared by covalently coupling the antidandruff agent climbazole with a skin-binding peptide using a two step procedure. First, climbazole is reacted with epichlorohydrin to introduce an epoxide group into the molecule. Then, the epoxide adduct is reacted with a lysine-capped, skin-binding peptide.

### Preparation of 1-(4-chlorophenyl)-1-(12-dihydro-1-((oxiran-2-yl)methyl) imidazol-3-yl)-3,3-dimethylbutan-2-one

Climbazole (I) (CAS 38083-17-9), 1 g, 3.6 mmol, is dissolved in benzene (100 mL). Epichlorohydrin, 0.67 g, 7.2 mmol, dissolved in 50 mL of dry acetonitrile is added with stirring and the mixture is heated at reflux for 72 h to prepare the 1-(4-chlorophenyl)-1-(1,2-dihydro-1-((oxiran-2-yl)methyl)imidazol-3-yl)-3,3-dimethylbutan-2-one adduct (II).

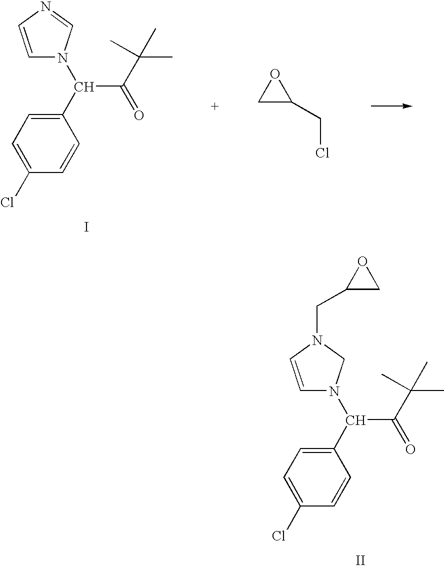

The epoxide adduct (II) is isolated by vacuum distillation of the solvent and the resulting intermediate is recrystalized from a suitable organic solvent mixture selected from ethyl acetate, toluene, acetonitrile, methyl ethyl ketone or chloroform.

### Coupling of Epoxide Adduct to Skin-Binding Peptide:

The purified epoxide adduct (II), 1 g, 3 mmol is then dissolved in dry dimethylformamide (DMF) or other suitable solvent (50 mL) and to this solution is added the lysine-capped, skin binding peptide, given as SEQ ID NO:22 (Synpep; Dublin, Calif.), 0.25 to 5 molar equivalents, also dissolved in DMF or other suitable solvent. A trace amount of water is added to facilitate opening the epoxide ring. Triethylamine is added to ensure that the amine groups on the skin-binding peptide are sufficiently activated for reaction with the epoxide group. The resultant reaction mixture is stirred and optionally heated up to several hours to couple the epoxide adduct to the peptide via reaction of the epoxide group with the terminal amine groups on the skin binding peptide. The peptide-based antidandruff reagent is isolated by vacuum distillation of the solvent and purified using chromatographic techniques.

This method can also be used to prepare peptide-based antidandruff reagents comprising other azole antidandruff agents, such as itraconazole and ketoconazole.

### Example 2 (Prophetic)

### Preparation of an Antidandruff Shampoo Comprising a Peptide-Based Antidandruff Reagent

The purpose of this prophetic Example is to describe the preparation of an antidandruff shampoo composition comprising a peptide-based antidandruff reagent.

The antidandruff shampoo composition is prepared using the ingredients listed in Table 1.

TABLE 1
Antidandruff Shampoo Composition

| Ingredient | Wt % |
|---|---|
| Ammonium Laureth Sulfate | 12 |
| Sodium Laureth Sulfate | 5 |
| Dihydrogenated tallow phthalic acid | 4 |
| amide | |
| Cocamide MEA | 2 |
| Polyquaternium-10 | 1 |
| Peptide-based antidandruff reagent, | 0.5 |
| prepared as described in Example 1 | |
| Citric acid | to adjust pH |
| Disodium EDTA | 0.5 |
| Fragrance | 0.7 |
| Water | qs to 100 |

The shampoo composition is prepared by combining water and the EDTA, heating to 65° C. and mixing until the EDTA is dissolved. Then the remaining ingredients are added, and the mixture is mixed until all the solids are dissolved. The pH is adjusted with citric acid as desired.

### Example 3 (Prophetic)

### Preparation of an Antidandruff Conditioner Comprising a Peptide-Based Antidandruff Reagent

The purpose of this prophetic Example is to describe the preparation of an antidandruff conditioner composition comprising a peptide-based antidandruff reagent.

The antidandruff conditioner composition is prepared using the ingredients listed in Table 2.

TABLE 2
Antidandruff Conditioner Composition

| CFTA Names | Wt % |
|---|---|
| Self emulsifying glyceryl ester | 6.0 |
| Cetrimonium chloride | 3.5 |
| Dicetyldimonium chloride | 3.0 |
| Cetearyl alcohol | 2.0 |
| Peptide-based antidandruff reagent from Example 1 | 0.3 |
| Trimethylsyllamodimethicone | 0.7 |
| Menthol | 0.1 |
| Phytolipid and hyaluronic Acid | 0.1 |
| Apricot seed (Apricot Kernel Powder produced by | 0.25 |
| Alban Muellen, Inc. of Paris, France) | |
| Pearlizing agent | 0.8 |
| Methyl gluceth-20 | 0.25 |
| Polyquaternium-4 | 0.1 |
| Water | qs to 100 |

To 55 g of deionized water heated to 60° C., the first 4 ingredients are added serially with moderate agitation until completely dissolved. The bulk solution is then cooled to 35° C., and the remaining ingredients are added serially with moderate agitation.

What is claimed is:

1. A peptide-based antidandruff reagent having the general structure (SBPₘ)ₙ−(ADA)_{y}, wherein
a) SBP is a skin-binding peptide;
b) ADA is an antidandruff agent;
c) m ranges from 1 to about 100;
d) n ranges from 1 to about 100;
e) y ranges from 1 to about 100; and optionally comprising a spacer.

2. The peptide-based antidandruff reagent of claim 1, wherein the skin-binding peptide is from about 7 to about 35 amino acids in length.

3. The peptide-based antidandruff reagent of claim 1, wherein the skin-binding peptide is generated combinatorially by a process selected from the group consisting of phage display, yeast display, ribosome display, bacterial display, and mRNA-display or identified empirically.

4. The peptide-based antidandruff reagent of claim 1, wherein the skin-binding peptide is selected from the group consisting of SEQ ID NOs:1, 2, 3, 4, 5, 6, 7, 8, 9, 10, 11, 12, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, and 58.

5. The peptide-based antidandruff reagent of claim 1, wherein the skin-binding peptide further comprises a cysteine or lysine residue on at least one end of the peptide selected from the group consisting of:
a) the N-terminal end; and
b) the C-terminal end.

6. The peptide-based antidandruff reagent of claim 1, wherein the skin-binding peptide further comprises a proline or aspartic acid residue on at least one end of the peptide selected from the group consisting of
a) the N-terminal end; and
b) the C-terminal end.

7. The peptide-based antidandruff reagent of claim 1, wherein the antidandruff agent is selected from the group consisting of a calcium pyridinethione salt, a magnesium pyridinethione salt, a barium pyridinethione salt, a strontium pyridinethione salt, a zinc pyridinethione salt, a zirconium pyridinethione salt, piroctone olamine, climbazole, ketoconazole, itraconazole, undecylenic acid, undecylenamidopropylbetaine, and combinations of these.

8. The peptide-based antidandruff reagent according to claim 9 wherein the zinc pyrithione salt is the zinc salt of 1-hydroxy-2-pyridinethione.

9. The peptide-based antidandruff reagent of claim 1, wherein the antidandruff agent is an antifungal peptide selected from the group consisting of syringomycins, syringostatins, syringotoxins, nikkomycins, echinocandins, pneumocadins, aculeacins, mulundocadins, cecropins, α-defensins, β-defensins, novispirins, and mixtures thereof.

10. The peptide-based antidandruff reagent of claim 1, wherein the skin-binding peptide is identified by a process comprising the steps of:
(a) providing a combinatorial library of DNA associated peptides;
(b) contacting the library of (a) with a skin sample to form a reaction solution comprising DNA associated peptide-skin complexes;
(c) isolating the DNA associated peptide-skin complexes of (b);
(d) amplifying the DNA encoding the peptide portion of the DNA associated peptide-skin complexes of (c); and
(e) sequencing the amplified DNA of (d) encoding a skin-binding peptide, wherein the skin-binding peptide is identified.

11. The peptide-based antidandruff reagent of claim 10, wherein after step (c):
i) the DNA associated peptide-skin complexes are contacted with an eluting agent whereby a portion of DNA associated peptides are eluted from the skin and a portion of the DNA associated peptides remain complexed; and
ii) the eluted or complexed DNA associated peptides of (i) are subjected to steps (d) and (e).

12. The peptide-based antidandruff reagent of claim 10, wherein the DNA encoding the peptides is amplified by a process selected from the group consisting of:
i) amplifying DNA comprising a peptide coding region by polymerase chain reaction; and
ii) infecting a host cell with a phage comprising DNA encoding the peptide and growing said host cell in a suitable growth medium.

13. The peptide-based antidandruff reagent of claim 12, wherein the peptides encoded by the amplified DNA of step (d) are contacted with a fresh skin sample and steps (b) through (d) are repeated one or more times.

14. The peptide-based antidandruff reagent according to claim 1, wherein the spacer is a peptide comprising amino acids selected from the group consisting of proline, lysine, glycine, alanine, serine, and mixtures of these.

15. The peptide-based antidandruff reagent according to claim 14, wherein the peptide spacer comprises an amino acid sequence selected from the group consisting of SEQ ID NOs: 13, 14, 15, and 16.

16. The peptide-based antidandruff reagent of claim 1, wherein the spacer is selected from the group consisting of ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, ethyl alkyl chain, propyl alkyl chain, hexyl alkyl chain, steryl alkyl chains, cetyl alkyl chains, and palmitoyl alkyl chains.

17. A hair care composition comprising an effective amount of the peptide-based antidandruff reagent of claim 1.

18. The hair care composition according to claim 17, wherein said composition is selected from the group consisting of a shampoo, a conditioner, a rinse, a lotion, an aerosol, a gel, a mousse, and a hair dye.

19. The hair care composition according to claim 17, wherein the composition further comprises at least one cosmetic raw material or adjuvant selected from the group consisting of antioxidants, preserving agents, fillers, surfactants, UVA and/or UVB sunscreens, fragrances, viscosifying agents, wetting agents, anionic polymers, nonionic polymers, amphoteric polymers, viscosity/foam stabilizers, opacifying/pearlizing agents, sequestering agents, stabilizing agents, hair conditioning agents, humectants, anti-static agents, anti-freezing agents, buffering agents, dyes, and pigments.

20. A method for treating or preventing dandruff comprising the steps of:
a) providing a hair care composition comprising a peptide-based antidandruff reagent selected from the group consisting of:
(SBPₘ)ₙ−(ADA)_{y}; and i)
[(SBP)ₓ−S_{m]}ₙ−(ADA)_{y} ii)
wherein
1) SBP is a skin-binding peptide;
2) ADA is an antidandruff reagent agent;
3) n ranges from 1 to about 100;
4) S is a spacer;
5) m ranges from 1 to about 100;
8) x ranges from 1 to about 10; and
9) y ranges from 1 to about 100;
and wherein the skin binding peptide is selected by a method comprising the steps of:
A) providing a combinatorial library DNA associated peptides;
B) contacting the library of (A) with a skin sample to form a reaction solution comprising DNA associated peptide-skin complexes;
C) isolating the DNA associated peptide-skin complexes of (B);
D) amplifying the DNA encoding the peptide portion of the DNA associated peptide-skin complexes of (C); and
E) sequencing the amplified DNA of (d) encoding a skin-binding peptide, wherein the skin-binding peptide is identified; and
b) applying the hair care composition of (a) to the scalp.

21. The method of claim 20, wherein after step (C):
i) the DNA associated peptide-skin complexes are contacted with an eluting agent whereby a portion of DNA associated peptides are eluted from the skin and a portion of the DNA associated peptides remain complexed; and
ii) the eluted or complexed DNA associated peptides of (i) are subjected to steps (D) and (E); and
wherein the DNA encoding the peptides is amplified by a process selected from the group consisting of:
a) amplifying DNA comprising a peptide coding region by polymerase chain reaction; and
b) infecting a host cell with a phage comprising DNA encoding the peptide and growing said host cell in a suitable growth medium.

22. The method according to claim 21, wherein the peptides encoded by the amplified DNA of step (D) are contacted with a fresh skin sample and steps (B) through (D) are repeated one or more times.
